(19) United States
(12) Patent Application Publication
(10) Pub. No.: US 2015/0225484 A1
(43) Pub. Date: Aug. 13, 2015

(54) ANTIGEN-BINDING MOLECULE AND USES THEREOF

(71) Applicant: Affimed GmbH, Heidelberg (DE)

(72) Inventors: Melvyn LITTLE, St. Peter-Ording (DE); Fabrice LE GALL, Edingen-Neckarhausen (DE)

(21) Appl. No.: 14/696,754

(22) Filed: Apr. 27, 2015

Related U.S. Application Data
(62) Division of application No. 13/727,059, filed on Dec. 26, 2012.
Application No. 13/727,059 is a division of application No. 13/034,920, filed on Feb. 25, 2011.
(60) Provisional application No. 61/308,205, filed on Feb. 25, 2010.

(51) Int. Cl. C07K 16/30; C07K 16/28

(52) U.S. Cl. CPC C07K 16/30; C07K 16/2803; C07K 16/2896; C07K 2317/626; C07K 2317/73; C07K 2317/31; C07K 2317/24; C07K 2317/92

(57) ABSTRACT

In one aspect, the present invention relates to an antigen-binding molecule specific for albumin and CD3 which may comprise two polypeptide chains, each polypeptide chain having at least four variable domains in an orientation preventing Fv formation and the two polypeptide chains are dimerized with one another thereby forming a multivalent antigen-binding molecule. On each of the two polypeptide chains the four variable domains may be arranged in the order V_{L}A-V_{H}B-V_{L}B-V_{H}A from the N-terminal to the C-terminal of the polypeptide. Compositions of the antigen-binding molecule and the methods of using the antigen-binding molecule or the compositions thereof for treatment of various diseases are also provided herein.

### RELATED APPLICATIONS AND INCORPORATION BY REFERENCE

This application is a divisional application of U.S. patent application Ser. No. 13/727,059 filed Dec. 26, 2012 which is a divisional application of U.S. patent application Ser. No. 13/034,920 filed Feb. 25, 2011 which claims priority to U.S. provisional patent application Ser. No. 61/308,205 filed Feb. 25, 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to an antigen-binding molecule specific for albumin and CD3, compositions of the antigen-binding molecule and the methods of using the antigen-binding molecule or the compositions thereof for treatment of various diseases.

### BACKGROUND OF THE INVENTION

Various formats of multivalent recombinant antibody fragments have been designed as alternatives to quadroma derived antibodies.

U.S. Pat. No. 7,129,330 and Kipriyanov et al. J. Mol. Biol. (1999) 293, 41-56 relates to the construction and production of a particular format of multivalent antibody fragments which are named “tandem diabodies” (TandAb®), since their design is based on intermolecular pairing of V_{H} and V_{L} variable domains of two different polypeptides as described for diabodies (Holliger et al., 1993, Proc. Natl. Acad. Sci. USA, 90:6444-6448). The antibodies are bispecific for CD19 and CD3. In contrast to bivalent scFv-scFv (scFv)₂ tandems the tandem diabodies are tetravalent, because they have four antigen-binding sites. Polypeptides with the domain order V_{H}A-V_{L}B-V_{H}B-V_{L}A from the N-terminus to the C-terminus of the polypeptides forming the tandem diabodies are portrayed. The orders of variable domains and the linker peptides between them were designed such that each domain associates with a complementary domain in another identical molecule thereby forming the dimerized tetravalent tandem diabodies. The tandem diabodies are devoid of immunoglobulin constant domains. It was reported that the tandem diabodies have advantages such as a high affinity, a higher avidity, lower clearance rates and exhibit a favorable in vitro and in vivo efficiency.

Several additional tandem diabodies are known comprising antibody specificities such as, for example, anti-CD16, anti-EpCAM and anti-CD30. In all cases, however, the order of the four antibody domains along the polypeptide chains of the tandem diabody from the N-terminus to the C-terminus was always V_{H}A-V_{L}B-V_{H}B-V_{L}A, where V_{H} and V_{L} represent the antibody heavy and light chain variable domains of antibodies with specificities for antigens A and B, respectively.

Such bispecific tandem diabodies can make a bridge between a tumor cell (e.g. B-CLL cell) and an effector cell of the human immune system (NK cell, T cell, monocyte, macrophage or granulocyte) thus permitting killing of the tumour cell. The tight binding of the tumor cell and the cytotoxic cell induces the destruction of the tumor cell.

While such tandem diabodies have proved to be favorable for therapeutic applications, e.g. for therapeutic concepts for the treatment of tumors, there remains a need for improved antigen-binding molecules.

Citation or identification of any document in this application is not an admission that such document is available as prior art to the present invention.

### SUMMARY OF THE INVENTION

In one aspect, the present invention provides a dimeric antigen-binding molecule comprising a first and a second polypeptide chain, each of the first and the second polypeptide chains which may comprise (a) a first domain V_{L}A being a light chain variable domain specific for a first antigen A; (b) a second domain V_{H}B being a heavy chain variable domain specific for a second antigen B; (c) a third domain V_{L}B being a light chain variable domain specific for the second antigen B; and (d) a fourth domain V_{H}A being a heavy chain variable domain specific for the first antigen A, wherein said domains may be arranged in each of said first and second polypeptide chains in the order V_{L}A-V_{H}B-V_{L}B-V_{H}A from the N-terminus to the C-terminus of said polypeptide chains, and the first domain V_{L}A of the first polypeptide chain may be in association with the fourth domain V_{H}A of the second polypeptide chain to form an antigen binding site for the first antigen A; and the second domain V_{H}B of the first polypeptide chain may be in association with the third domain V_{L}B of the second polypeptide chain to form an antigen binding site for the second antigen B; and the third domain V_{L}B of the first polypeptide chain may be in association with the second domain V_{H}B of the second polypeptide chain to form an antigen binding site for the second antigen B; and the fourth domain V_{H}A of the first polypeptide chain may be in association with the first domain V_{L}A of the second polypeptide chain to form an antigen binding site for the first antigen A.

In some embodiments, the antigen-binding molecule as described herein may be a homodimer and the first and the second polypeptide chains have the same amino acid sequence. In some embodiments, the first and the second polypeptide chains may be non-covalently associated. In some embodiments, the antigen-binding molecule may be tetravalent. In some embodiments, the antigen-binding molecule may be bispecific. In some embodiments, the domains may be human domains or humanized domains. In some embodiments, the antigen-binding molecule may comprise at least one further functional unit. In some embodiments, the antigen binding molecule may be specific for a B-cell, T-cell, natural killer (NK) cell myeloid cell or phagocytotic cell. In some embodiments, the antigen-binding molecule may be bispecific, which antigen-binding molecule may be further specific for a tumor cell. In some embodiments, the first light chain variable domain (V_{L}A) and the first heavy chain variable domain (V_{H}A) may be specific for a tumor cell. In some embodiments, the antigen-binding molecule may be bispecific for serum albumin and CD3.

In another aspect, the present invention provides a polypeptide chain which may comprise (a) a first domain V_{L}A being a light chain variable domain specific for a first antigen A; (b) a second domain V_{H}B being a heavy chain variable domain specific for a second antigen B; (c) a third domain V_{L}B being a light chain variable domain specific for the second antigen B; and (d) a fourth domain V_{H}A being a heavy chain variable domain specific for the first antigen A; wherein the domains are arranged in the polypeptide chain in the order V_{L}A-V_{H}B-V_{L}B-V_{H}A from the N-terminus to the C-terminus of the polypeptide chains. In some embodiments, the first domain V_{L}A and the fourth domain V_{H}A may not associate to form an antigen binding site for the first antigen A and the second domain V_{H}B and the third domain V_{L}B may not associate to form an antigen binding site for the second antigen B. In some embodiments, the first domain V_{L}A and the second domain V_{H}B, the second domain V_{H}B and the third domain V_{L}B, and the third domain V_{L}B and the fourth domain V_{H}A may be separated by not more than about 12 amino acid residues. In some embodiments the polypeptide chain may comprise amino acid residues upstream from the first domain V_{L}A and/or downstream from the fourth domain V_{H}A. In some embodiments, the polypeptide chain may be linked to a further functional unit. In a particular embodiment the variable domains may be specific for serum albumin and CD3.

In another aspect, the present invention provides a nucleic acid molecule encoding a polypeptide chain as described herein. In another aspect, the present invention provides a pharmaceutical composition comprising the antigen-binding molecule, the polypeptide chain or the nucleic acid molecule as disclosed herein and a pharmaceutically acceptable carrier.

In yet another aspect, the present invention provides a method for the treatment of an autoimmune disease, inflammatory disease, infectious disease, allergy and/or cancer which may comprise administering an effective amount of the antigen-binding molecule, the nucleic acid molecule or the composition of the present invention to a subject in need thereof.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The novel features of the invention are set forth with particularity in the appended claims. A better understanding of the features and advantages of the present invention will be obtained by reference to the following detailed description that set forth illustrative embodiments, in which the principles of the invention are utilized, and the accompanying drawings of which:

FIG. 1 illustrates the gene organization of a construct encoding an antigen-molecule according to the invention, where V_{L}A represents a light chain variable immunoglobulin domain specific for an antigen A, V_{H}B represents a heavy chain variable immunoglobulin domain specific for an antigen B, V_{L}B represents a light chain variable immunoglobulin domain specific for the antigen B, V_{H}A represents a heavy chain variable immunoglobulin domain specific for the antigen A, L1 a peptide linker or a peptide bond connecting V_{L}A and V_{H}B, L2 a peptide linker or a peptide bond connecting V_{H}B and V_{L}B, and L3 a peptide linker or a peptide bond connecting V_{L}B and V_{H}A.

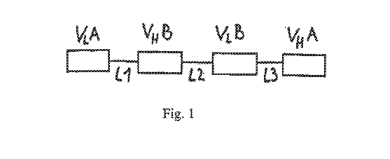

FIG. 2 illustrates the formation of a dimeric antigen-binding molecule according to the invention from non-functional monomeric polypeptide chains (A) by intra-molecular pairing of variable domains of a first polypeptide chain 1 and a second polypeptide chain 2 with one another (B) to a functional antigen-binding molecule according to the inventions in the format of a tandem diabody, where “1” represents the first polypeptide chain, “2” represents the second polypeptide chain, V_{L}A represents a light chain variable immunoglobulin domain specific for an antigen A, V_{H}B represents a heavy chain variable immunoglobulin domain specific for an antigen B, V_{L}B represents a light chain variable immunoglobulin domain specific for the antigen B, V_{H}A represents a heavy chain variable immunoglobulin domain specific for the antigen A, L1 a peptide linker or a peptide bond connecting V_{L}A and V_{H}B, L2 a peptide linker or a peptide bond connecting V_{H}B and V_{L}B, and L3 a peptide linker or a peptide bond connecting V_{L}B and V_{H}A.

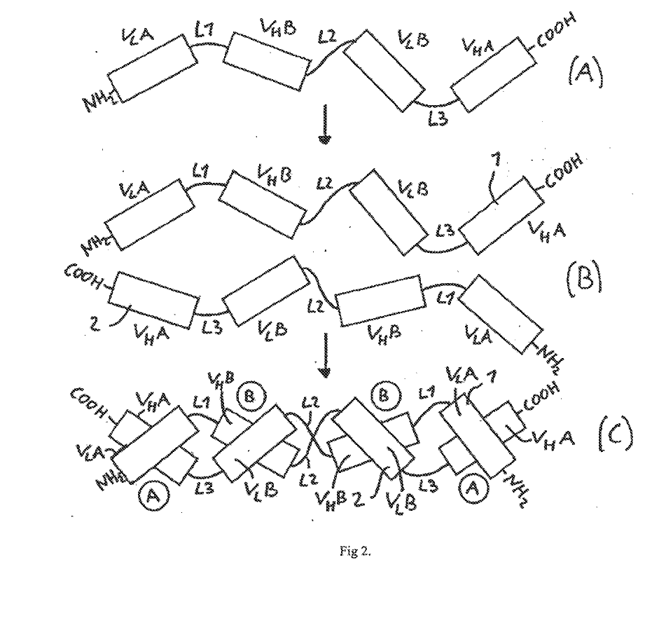

FIG. 3 shows a comparison of CD19×CD3 tandem diabodies in a cytotoxicity assay. Option 0=antibody A1 with the domain order V_{H}A-V_{L}B-V_{H}B-V_{L}A. Option 2 =antibody B with the domain order V_{L}A-V_{H}B-V_{L}B-V_{H}A according to the invention. 1×10⁴ calcein-labelled Raji cells were incubated with 5×10⁵ PBMC in the presence of increasing concentrations of the indicated CD19×CD3 tandem diabodies. PBMC were cultured overnight in the presence of 25 U/mL human IL-2 before they were used as effector cells in the assay. After 4 h incubation fluorescent calcein in the cell culture medium released from apoptotic target cells was measured at 520 nm and % specific lysis was calculated. EC₅₀ values were analysed by non-linear regression using GraphPad software. The mean and standard deviations of duplicates were plotted.

FIG. 4 shows a comparison of CD19×CD3 tandem diabodies in a cytotoxicity assay. Option 0=antibody A2 with the domain order V_{H}A-V_{L}B-V_{H}B-V_{L}A. Option 2 =antibody C with the domain order V_{L}A-V_{H}B-V_{L}B-V_{H}A according to the invention. 1×10⁴ calcein-labelled Raji cells were incubated with 5×10⁵ freshly isolated PBMC in the presence of increasing concentrations of the indicated CD19×CD3 tandem diabodies. After 4 h incubation fluorescent calcein in the cell culture medium released from apoptotic target cells was measured at 520 nm and % specific lysis was calculated. EC₅₀ values were analysed by non-linear regression using GraphPad software. The mean and standard deviations of duplicates were plotted.

FIG. 5 shows the TCR modulation by HSA×CD3 TandAb antibodies in the presence or absence of HSA. CD3⁺ Jurkat cells were cultured for 2 h in the presence of increasing concentrations of the HSA×CD3 TandAb option 0 (V_{H}A-V_{L}B-V_{H}B-V_{L}A; triangle) or option 2 (V_{L}A-V_{H}B-V_{L}B-V_{H}A according to the invention; square) antibodies with (filled symbols) or without (open symbols) the addition of 50 mg/mL HSA. After washing, remaining TCR/CD3 complexes were measured by flow cytometry using a PC5-conjugated anti-TCRα/β antibody. Mean fluorescence values were used for analysis by non-linear regression (experiment CAB-306).

FIG. 6 shows the vector map with the restriction sites of pCDNA5FRT which encodes antibody B.VH and VL: variable domains of the heavy and the light chains.

FIG. 7 shows the vector map with the restrictions sites of pSKK3 which encodes antibody C. VH and VL: variable domains of the heavy and light chains.

### DETAILED DESCRIPTION OF THE INVENTION

In one aspect, the present invention provides a recombinant dimeric and tetravalent antigen-binding molecule with four immunoglobulin domains (two heavy chain variable domains and two light chain variable domains) linked with one another in a polypeptide chain and arranged in the order V_{L}A-V_{H}B-V_{L}B-V_{H}A from the N-terminus to the C-terminus of the polypeptide chain. Such an antigen-binding molecule of the present invention triggers an enhanced biological activity, such as, e.g., an enhanced immune response or enhanced immune suppression.

In one embodiment, it illustrates that a dimeric, bispecific antigen-binding molecule of the tandem diabody format being specific for CD3 and CD19 and having polypeptide chains with the domain order V_{L}A-V_{H}B-V_{L}B-V_{H}A is more than 60 times more active in vitro, i.e. cytotoxic, than a corresponding tandem diabody molecule with the same domains but in the reverse domain order V_{H}A-V_{L}B-V_{H}B-V_{L}A.

In another embodiment, it illustrates that a dimeric, bispecific antigen-binding molecule of the tandem diabody format being specific for albumin (HSA) and CD19 and having polypeptide chains with the domain order V_{L}A-V_{H}B-V_{L}B-V_{H}A has a significantly more effective T cell receptor modulation activity in vitro, i.e. is more immunosuppressive, than a corresponding tandem diabody molecule with the same domains but in the reverse domain order V_{H}A-V_{L}B-V_{H}B-V_{L}A.

Thus, tandem diabodies with the domain order V_{L}A-V_{H}B-V_{L}B-V_{H}A from the N-terminus to the C-terminus of the polypeptide chains have an increased potential for immunotherapy. A further advantage of the enhanced biological activity is that the effective therapeutic dosages for such tandem diabodies may be reduced. Moreover, side effects caused by the administered antigen binding molecules may also be reduced due to the lower dosages. Without being bound by any theory, the new domain order allows a modified crosslinking of the dimeric antigen binding molecule between the antigen A and the antigen B compared with the tandem diabodies of the art and, in certain aspects of the invention, this will enable the molecule to bind to target antigens, e.g., receptors, more efficiently than the dimeric antigen binding molecules of the art.

Therefore, the biological activity of a dimeric, antigen-binding molecule such as a tandem diabody can be enhanced, when the four variable domains of each polypeptide chain which form the dimeric antigen-binding molecule are arranged in the order V_{L}A-V_{H}B-V_{L}B-V_{H}A from the N-terminus to the C-terminus of each polypeptide chain. The triggered “biological activity” depends on the specificities of the antigen-binding molecule and may encompass cytotoxicity, phagocytosis, antigen presentation, cytokine release or immune suppression, in particular antibody dependent cell mediated cytotoxicity (ADCC), antibody dependent cell mediated phagocytosis (ADCP)and/or complement dependent cytotoxicity (CDC).

In some embodiments, the present invention provides a dimeric antigen-binding molecule comprising a first and a second polypeptide chain, wherein each of the first and the second polypeptide chains comprises a first domain V_{L}A being a light chain variable domain specific for a first antigen A, a second domain V_{H}B being a heavy chain variable domain specific for a second antigen B, a third domain V_{L}B being a light chain variable domain specific for the second antigen B, a fourth domain V_{H}A being a heavy chain variable domain specific for the first antigen A, and said domains are arranged in each of said first and second polypeptide chains in the order V_{L}A-V_{H}B-V_{L}B-V_{H}A from the N-terminus to the C-terminus of said polypeptide chains.

In some embodiments, the first, second, third and fourth variable domains are arranged in an orientation preventing intramolecular pairing within the same polypeptide chain and the first polypeptide chain is associated, i.e. dimerized, with the second polypeptide chain such that the first domain V_{L}A of the first polypeptide chain is in association with the fourth domain V_{H}A of the second polypeptide chain to form an antigen binding site for the first antigen A, the second domain V_{H}B of the first polypeptide chain is in association with the third domain V_{L}B of the second polypeptide chain to form an antigen binding site for the second antigen B, the third domain V_{L}B of the first polypeptide chain is in association with the second domain V_{H}B of the second polypeptide chain to form an antigen binding site for the second antigen B and the fourth domain V_{H}A of the first polypeptide chain is in association with the first domain V_{L}A of the second polypeptide chain to form an antigen binding site for the first antigen A.

The term “antigen-binding molecule” refers to an immunoglobulin derivative with multivalent antigen-binding properties, preferably having at least four antigen-binding sites. Each antigen-binding site is formed by a heavy chain variable domain V_{H} and a light chain variable domain V_{L} of the same antigen, i.e. epitope, specificity. Preferably the antigen-binding molecule according to the invention is devoid of immunoglobulin constant domains or fragments of immunoglobulin constant domains, but in certain cases described below a constant domain or parts thereof may be linked to the antigen-binding molecule.

The antigen-binding molecule is “dimeric” which term refers to a complex of two polypeptide monomers. These two polypeptide monomers are the first and the second polypeptide chains. Preferably the antigen-binding molecule is a “homodimer” which term means that the antigen-binding molecule is composed of identical polypeptide monomers. In a preferred homodimeric antigen-binding molecule according to the invention the first and the second polypeptide chain may have the same amino acid sequence, i.e. the first and the second polypeptide chains are identical and, thus, are encoded and expressed by the same single polynucleotide. This is different in the case of so-called bispecific diabodies, which are heterodimers that are encoded by two distinct polynucleotides. In the former case each of the first and the second polypeptide chains contain four variable domains, four binding sites are formed and the antigen-binding molecule is tetravalent. Such tetravalent homodimeric antigen-binding molecules have received some recognition in the art as tandem diabodies.

Preferably, in the antigen-binding molecule the first and the second polypeptide chain are non-covalently associated with each other, in particular with the proviso that there is no covalent bond between the first and second polypeptide chain. However, if desired, the two polypeptide chains may be additionally stabilized by at least one covalent linkage, e.g. by a disulfide bridge between cysteine residues of different polypeptide chains.

The term “polypeptide chain” refers to a polymer of amino acid residues linked by amide bonds. The first and the second polypeptide chains are, preferably, single chain fusion proteins which are not branched. In each of the first and second polypeptide chains the four domains are arranged such that the second domain V_{H}B is C-terminal from the first domain V_{L}A, the third domain V_{L}B is C-terminal from the second domain V_{H}B and the fourth domain V_{H}A is C-terminal from the third domain V_{L}B. The first and the second polypeptide chains may have contiguous amino acid residues in addition N-terminal to the first domain V_{L}A and/or C-terminal to the fourth domain V_{H}A. For example, the polypeptide chain may contain a Tag sequence, preferably at the C-terminus which might be useful for the purification of the polypeptide. An example of a Tag sequence is a His-Tag, e.g. a His-Tag consisting of six His-residues.

In some embodiments, the first, second, third and fourth domains are covalently connected such that the domains of the same polypeptide chain do not associate, i.e. pair, with each other. The domains may be linked such that the first domain V_{L}A is linked with the second domain V_{H}B by a first linker L1, the second domain V_{H}B is linked with the third domain V_{L}B by a second linker L2 and the third domain V_{L}B is linked with the fourth domain V_{H}A by a third linker L3, wherein the first linker L1 and the third linker L3 are distal to the central linker L2 on each of the first and second polypeptide chains. Linker L1, linker L2 and linker L3 can be each a peptide linker comprising at least one amino acid residue or a peptide bound without any intervening amino acid residue between the two adjacent domains.

In some embodiments, the length of each of the linkers L1, L2 and L3 is such that the domains of the first polypeptide chain can associate with the domains of the second polypeptide chain to form the dimeric antigen-binding molecule. The length of the linkers influences the flexibility of the antigen-binding molecule. The desired flexibility of the antigen-binding molecule depends on the target antigen density and the acessibility of the target antigen, i.e. epitopes. Longer linkers provide more flexible antigen-binding molecules with more agile antigen-binding sites. The effect of linker length on the formation of dimeric antigen-binding molecules is described, for example, in Todorovska et al., 2001 Journal of Immunological Methods 248:47-66; Perisic et al., 1994 Structure 2:1217-1226; Le Gall et al., 2004, Protein Engineering 17:357-366 and WO 94/13804.

In certain preferred embodiments, the linkers L1, L2 and/or L3 are “short”, i.e. consist of 0, 1, 2, 3, 4, 5, 6, 7, 8, 9, 10, 11 or about 12 amino acid residues. Such short linkers favor the correct dimerization of the first with the second polypeptide chain by binding and forming antigen-binding sites between light chain variable domains and heavy chain variable domains of different polypeptide chains. In particular, the central linker L2 should be short such that it prevents formation of a single chain Fv (scFv) antigen-binding unit within the same polypeptide chain by the two adjacent domains V_{H}B and V_{L}B. The central linker L2 influences the flexibility of the polypeptide chain. If the central linker L2 is long, and flexible (in general consisting of about 12 or more amino acid residues) the polypeptide chain can fold head-to-tail and form a single-chain antigen-binding molecule known in the art as a single chain diabody. If the central linker L2 is short and rigid the polypeptide chain cannot fold head-to-tail and dimerizes with another polypeptide chain. Shortening the linker to about 12 or less amino acid residues generally prevents adjacent domains of the same polypeptide chain from interacting with each other. Therefore, the central linker L2 and the distal linkers L1 and L3 should preferably consist of about 12 or less amino acid residues to prevent pairing of adjacent domains of the same polypeptide chain. In a preferred embodiment of the invention the linkers L1, L2 and/or L3 consist of about 3 to about 10 contiguous amino acid residues. The linkers may consist of different numbers of amino acid residues, but it is preferred that the distal linkers L1 and L3 have the same number of amino acid residues or do not differ in length by more than one or two amino acid residues. In a certain aspect of the invention at least one of the linkers L1, L2 and/or L3 consists of nine amino acid residues. In a particular embodiment of the invention all three linkers L1, L2 and L3 consist of nine amino acid residues. In some embodiments, at least one of the linkers L2, L2 and/or L3 consists of less than between 10 to 3 amino acid residues.

Additional amino acid residues provide extra flexibility. In an alternative aspect the central linker L2 may have about 12 or less amino acid residues to prevent a head-to-tail folding of the polypeptide chain and at least one of the distal linkers L1 and/or L3 may have more than about 12 amino acid residues to provide extra flexibility. In another embodiment, two polypeptide chains having a central linker L2 with more than 12 amino acid residues correctly dimerize with one another to a tetravalent, dimeric antigen-binding molecule (see for example Le Gall et al., 2004, Protein Engineering 17:357-366). However, if longer linkers, e.g. consisting of about 13 or more, in particular of about 15 or more, amino acid residues are utilized, the dimeric antigen-binding molecule may be stabilized additionally by at least one covalent bond between such two polypeptide chains.

Regarding the amino acid composition of the linkers, in some embodiments, peptides are selected that do not interfere with the dimerization of the first and second polypeptide chains. For example, linkers comprising glycine and serine residues generally provide flexibility and protease resistance. The amino acid sequence of the linkers can be optimized, for example, by phage-display methods to improve the antigen binding and production yield of the molecules. In particular embodiments of the invention the linker may comprise the amino acid sequence GGSGGSGGS.

The first domain V_{L}A, the second domain V_{H}B, the third domain V_{L}B and the fourth domain V_{H}A are light chain and heavy chain variable domains of an immunoglobulin. The variable domains comprise the hypervariable loops or complementary binding regions (CDRs) containing the residues in contact with the antigen and the segments which contribute to the correct folding and display of the CDRs. It is preferred that each of the heavy chain and light chain variable domains comprises the respective three CDRs. The domains may be derived from any immunoglobulin class, e.g., IgA, IgD, IgE and IgM or a subclass thereof. The immunoglobulin may be of animal, in particular mammal, origin. Each domain may be a complete immunoglobulin heavy or light chain variable domain, a mutant, fragment or derivative of a naturally occurring variable domain, or a synthetic, e.g. recombinant domain which is genetically engineered. A derivative is a variable domain which differs by the deletion, substitution, addition or insertion of at least one amino acid from the amino acid sequence of a naturally occurring variable domain. Synthetic, e.g. recombinant domains, can be obtained, for example, by well known reproducible methods from hybridoma-derived antibodies or phage-display immunoglobulin libraries. For example phage display methods can be used to obtain variable domains of human antibodies to an antigen by screening libraries from human immunoglobulin sequences. The affinity of initially selected antibodies can be further increased by affinity maturation, for example chain shuffling or random mutagenesis. A person of ordinary skill in the art is familiar with methods for obtaining domains from natural or recombinant antibodies (for laboratory manuals see, for example, Antibody engineering: methods and protocols/edited by Benny K. C. Lo; Benny K. C. II Series: Methods in molecular biology (Totowa, N.J.)). Generally, any antibody known in the art can be used as a source for the variable domains of the invention.

In a certain aspect of the invention at least one, preferably all, of the first domain V_{L}A, the second domain V_{H}B, the third domain V_{L}B and the fourth domain V_{H}A are fully human, humanized or chimeric domains. A humanized variable domain comprises a framework region substantially having the amino acid sequence of a human immunoglobulin and a CDR of a non-human immunoglobulin. Humanized antibodies can be produced by well-established methods such as, for example CDR-grafting (see, for example, Antibody engineering: methods and protocols/edited by Benny K. C. Lo; Benny K. C. II Series: Methods in molecular biology (Totowa, N.J.)). Thus, a skilled person is readily able to make a humanized or fully human version of antigen-binding molecules and variable domains from non-human, e.g. murine, sources with the standard molecular biological techniques known in the art for reducing the immunogenicity and improving the efficiency of the antigen-binding molecule in a human immune system. In a preferred embodiment of the invention all domains (e.g. V_{L}A, V_{H}B, V_{L}B and V_{H}A) are humanized or fully human; most preferred, the dimeric antigen-binding molecule according to the invention is humanized or fully human. The term “Fully human” as used herein means that the amino acid sequences of the variable domains and the peptides linking the variable domains in the first and second polypeptide chains originate or can be found in humans. In certain embodiments of the invention the variable domains may be human or humanized but not the peptides linking the variable domains.

In one embodiment the first domain V_{L}A, the second domain V_{H}B, the third domain V_{L}B and the fourth domain V_{H}A are specific for the same antigen such that antigen-binding sites formed by the domains bind either to the same epitope or to different epitopes on the same antigen. In this case the expressions “antigen A” and “antigen B” refer to the same antigen. Such antigen-binding molecules are monospecific.

In another embodiment the first domain V_{L}A, the second domain V_{H}B, the third domain V_{L}B and the fourth domain V_{H}A are specific for different antigens such that V_{L}A and V_{H}A form an antigen-binding site for an antigen A of a first specificity and V_{H}B and V_{L}B form an antigen-binding site for an antigen B of a second specificity. The different antigens may be associated with different kind of cells or represent different antigens of the same kind of cell. Such antigen-binding molecules according to the invention are bispecific.

In some embodiments, at least one antigen-binding site may be specific for a bacterial substance, viral protein, autoimmune marker or an antigen present on a particular cell such as a cell surface protein of a B-cell, T-cell, natural killer (NK) cell, myeloid cell, phagocytic cell, tumor cell.

In an aspect of the invention the dimeric antigen-binding molecule is bispecific comprising a first specificity for an effector cell and a second specificity for a target cell different from the effector cell. Such antigen-binding molecules are able to cross-link two cells and can be used to direct effector cells to a specific target. In another aspect of the invention the dimeric antigen-binding molecule may be bispecific for a target cell and a molecule selected from the group consisting of a drug, toxin, radionucleotide, enzyme, albumin and lipoprotein, naturally occurring ligands such as cytokines or chemokines. If the target molecule is albumin, the albumin or serum albumin may be selected from the group consisting of human, bovine, rabbit, canine and mouse.

“Effector cells” typically refer to cells of the immune system which can stimulate or trigger cytotoxicity, phagocytosis, antigen presentation, cytokine release. Such effector cells are, for example but not limited to, T cells, natural killer (NK) cells, granulocytes, monocytes, macrophages, dendritic cells, erythrocytes and antigen-presenting cells. Examples of suitable specificities for effector cells include but are not limited to CD2, CD3, CD5, CD28 and other components of the T-cell receptor (TCR) for T cells; CD16, CD38, CD44, CD56, CD69, CD335 (NKp46), CD336 (NKp44), CD337 (NKp30), NKp80, NKG2C and NKG2D for NK cells; CD18, CD64 and CD89 for granulocytes; CD18, CD64, CD89 and mannose receptor for monocytes and macrophages; CD64 and mannose receptor for dendritic cells; CD35 for erythrocytes. In certain aspects of the invention those specificities, i.e. cell surface molecules, of effector cells are suitable for mediating cell killing upon binding of a bispecific antibody to such cell surface molecule and, thereby, inducing cytolysis or apoptosis.

“Target cells” typically refers to the sites to which the effector cells should be directed to induce or trigger the respective biological, e.g. immune, response. Examples of target cells may be tumor cells or infectious agents such as viral or bacterial pathogens, for example dengue virus, herpes simplex, influenza virus, HIV or cells carrying autoimmune targets such as IL-2, an autoimmune marker or an autoimmune antigen.

In a preferred embodiment of the invention the dimeric antigen-binding molecule is bispecific for a tumor cell and an effector cell, in particular a T cell or a NK cell. Suitable specificities for tumor cells may be tumor antigens and cell surface antigens on the respective tumor cell, for example specific tumor markers. Such a bispecific dimeric antigen-binding molecule binds to both the tumor cell and the immune effector cell thereby triggering the cytotoxic response induced by the T cell or the NK cell. The term “tumor antigen” as used herein comprises tumor associated antigen (TAA) and tumor specific antigen (TSA). A “tumor associated antigen” (TAA) as used herein refers to a protein which is present on tumor cells, and on normal cells during fetal life (once-fetal antigens), and after birth in selected organs, but at much lower concentration than on tumor cells. A TAA may also be present in the stroma in the vicinity of the tumor cell but expressed at lower amounts in the stroma elsewhere in the body. In contrast, the term “tumor specific antigen” (TSA) refers to a protein expressed by tumor cells. The term “cell surface antigen” refers to any antigen or fragment thereof capable of being recognized by an antibody on the surface of a cell.

Examples of specificities for tumor cells include but are not limited to CD19, CD20, CD30, the laminin receptor precursor protein, EGFR1, EGFR2, EGFR3, Ep-CAM, PLAP, Thomsen-Friedenreich (TF) antigen, MUC-1 (mucin), IGFR, CD5, IL4-R alpha, IL13-R, FcεRI and IgE as described in the art.

In one embodiment the specificity for an effector cell may be CD3 or CD16 and the specificity for a tumor cell may be selected from CD19, CD20, CD30, the laminin receptor precursor, Ep-CAM, EGFR1, EGFR2, EGFR3, PLAP, Thomsen-Friedenreich (TF) antigen, MUC-1 (mucin), IGFR, CD5, IL4-R alpha, IL13-R, FcεRI and IgE. Particular examples of such antigen binding molecules are bispecific for CD3 and CD19 or CD16 and CD30.

In a certain aspect of the invention the first domain V_{L}A and the fourth domain V_{H}A have the specificity for a tumor cell and the other two domains, namely the second domain V_{H}B and the third domain V_{L}B, have the specificity for an effector cell, in particular T cell or NK cell. In one embodiment the first domain V_{L}A and the fourth domain V_{H}A have the specificity for a tumor cell and the other two domains, namely the second domain V_{H}B and the third domain V_{L}B, have the specificity for CD3 or CD16. In a certain embodiment thereof the first domain V_{L}A and the fourth domain V_{H}A have a specificity for CD19, CD20, the laminin receptor precursor, Ep-CAM, EGFR1, EGFR2, EGFR3, PLAP, Thomsen-Friedenreich (TF) antigen, MUC-1 (mucin), IGFR, CD5, IL4-R alpha, IL13-R, FcεRI and the other two domains, namely the second domain V_{H}B and the third domain V_{L}B, have a specificity for CD3.

In another aspect of the invention the first domain V_{L}A and the fourth domain V_{H}A have the specificity for an effector cell, in particular T cell or NK cell, and the other two domains, namely the second domain V_{H}B and the third domain V_{L}B, have the specificity for a tumor cell. In one embodiment the first domain V_{L}A and the fourth domain V_{H}A have the specificity for a CD3 or CD16 and the other two domains, namely the second domain V_{H}B and the third domain V_{L}B, have the specificity for a tumor cell. In a particular preferred embodiment the first domain V_{L}A and the fourth domain V_{H}A have the specificity for a CD3 and the other two domains, namely the second domain V_{H}B and the third domain V_{L}B, have the specificity for a tumor cell selected from the group consisting of CD19, CD20, CD30, the laminin receptor precursor, Ep-CAM, EGFR1, EGFR2, EGFR3, PLAP, Thomsen-Friedenreich (TF) antigen, MUC-1 (mucin), IGFR, CD5, IL4-R alpha, IL13-R, FcεRI and IgE.

CD3 antigen is associated with the T-cell receptor complex on T-cells. In the case where specificity for an effector cell is CD3, the binding of the dimeric antigen-binding molecule according to the invention to CD3 triggers the cytotoxic activity of T-cells. By bispecific binding of the dimeric antigen binding molecule to CD3 and to a target cell, e.g. tumor cell, cell lysis of the target cell may be induced. Dimeric antigen-binding molecules with a specificity towards CD3 and their production are known in the art (and described for example in Kipriyanov et al., 1999, Journal of Molecular Biology 293:41-56, Le Gall et al., 2004, Protein Engineering, Design & Selection, 17/4:357-366). Monospecific anti-CD3 antigen binding molecules are known for their immunosuppressive properties by binding to and modulating the T cell receptor (WO2004/024771). In one embodiment, the antigen-binding molecule according to the present invention is bispecific for CD3 and albumin for use as a immunosuppressive agent, e.g. in transplantation.

The CD16 (FcγIIIA) antigen is a receptor expressed on the surface of NK cells. NK cells possess an inherent cytoloytic activity and by bispecific binding of the dimeric antigen-binding molecule according to the invention to CD 16 the cytotoxic activity of NK cell towards the target cell can be triggered. An example of a bispecific antigen-binding molecule having specificity towards CD16 is described, for example, in Arndt et al., 1999, Blood, 94:2562-2568. In a particular embodiment of the invention at least one of the heavy chain or light chain variable domains are from an anti-CD16 antibody described in WO 2006/125668, in particular of antibodies which recognizes the CD16A isoform, but not the CD16B isoform.

Dimeric antigen-binding molecules according to the invention, wherein the tumor specificity is towards CD19 antigen may be used for immunotherapy of B-cell malignancies, because the CD19 antigen is expressed on virtually all B-lineage malignancies from lymphoblastic leukemia (ALL) to non-Hodgkin's lymphoma (NHL). In particular for the treatment of non-Hodgkin's lymphoma dimeric antigen-binding molecules having specificity towards CD19 or CD20 can be used. Dimeric antigen-binding molecules having specificity towards CD19 and their production are known in the art (and described, for example, in Cochlovius et al., 2000, Cancer Research 60:4336-4341).

Dimeric antigen-binding molecules according to the invention, wherein the tumor specificity is towards the laminin receptor or the laminin receptor precursor may be used, for example but not limited, for the treatment of B-cell chronic lymphocyte leukemia (B-CLL), non-Hodgkin's lymphoma, Hodgkin's lymphoma, lung cancer, colon carcinoma, mammary carcinoma, pancreatic carcinoma, prostate cancer, in particular in the condition of metastasizing cancer or minimal residual cancer. Antigen-binding molecules having specificity towards the laminin receptor precursor are described, for example, in Zuber et al., 2008, J. Mol. Biol., 378:530-539.

Dimeric antigen-binding molecules according to the invention wherein the tumor specificity is towards EGFR1 may be of particular use in the treatment of cancers wherein EGFR1 expression is up-regulated or altered, for example in cancers of the breast, bladder, head and neck, prostate, kidney, non-small cell lung cancer, colorectal cancer and glioma.

Dimeric antigen-binding molecules according to the invention wherein the tumor specificity is towards TF-antigen may be particularly useful in treating breast or colon cancer and/or liver metastases.

Dimeric antigen-binding molecules wherein the tumor specificity is towards CD30 may be particularly useful in treating Hodgkin's disease. Antigen-binding molecules having the specificity towards CD30 are described, for example, in Arndt et al., 1999, Blood, 94:2562-2568.

Dimeric antigen-binding molecules wherein the tumor specificity is towards the alpha chain of the IL4 receptor (IL4R alpha) may be particularly useful in treating solid tumors, in particular carcinomas of the breast, ovaries, renal system, head and neck, malignant melanoma and AIDS-related Kaposi's sarcoma. Dimeric antigen-binding molecules wherein at least one additional specificity is towards EGFR3/HER3 and/or EGFR2/neu may be particularly useful in treating breast cancer. Dimeric antigen-binding molecules wherein the tumor specificity is towards IGFR may be particularly useful in treating prostate cancer, colorectal cancer, ovarian cancer or breast cancer.

Dimeric antigen-binding molecules wherein the tumor specificity is towards CD5 may be particularly useful in treating chronic lymphocytic leukaemia.

Dimeric antigen-binding molecules wherein the tumor specificity is towards MUC-I may be particularly useful in the treatment of gastric cancer and ovarian cancer.

Dimeric antigen-binding molecules wherein the tumor specificity is towards EpCAM may be particularly useful in the treatment of carcinomas of the colon, kidney, and breast.

Dimeric antigen-binding molecules wherein the tumor specificity is towards PLAP may be of particular use in the treatment of ovarian or testicular cancer.

Dimeric antigen-binding molecules wherein the tumor specificity is towards OFA-iLR may be particularly useful in the treatment of metastatic tumors.

In a certain aspect of the invention the antigen binding molecule as described herein is dimeric and bispecific for CD3 and CD19 or the antigen-binding molecule is dimeric and bispecific for CD16 and CD19. In a particular embodiment thereof the first domain V_{L}A and the fourth domain V_{H}A are specific for CD3 and CD16, respectively, while the second domain V_{H}B and the third domain V_{L}B are specific for CD19. In both cases the first and second polypeptide chains each have the domain order V_{L}^{CD3}-V_{H}^{CD19}-V_{L}^{CD19}-V_{H}^{CD3} or V_{L}^{CD16}-V_{H}^{CD19}-V_{L}^{CD19}-V_{H}^{CD16} from the N-terminus to the C-terminus of the polypeptide chains. In a preferred embodiment the first, second, third and fourth domains are humanized or fully human. In a most preferred embodiment the first and second polypeptide chain as defined above is humanized or fully human. In another aspect of the invention the dimeric antigen binding molecule may be bispecific, for example, to EpCAM and CD3, albumin, such as, e.g., HSA and CD3 or EGFR and CD3.

A further aspect of the invention provides a dimeric antigen-binding molecule according to any one of the embodiments described above which is linked with a further functional unit, e.g. a functional domain or agent, which independently mediates a biological function, in particular a biochemical event. The further functional unit may be complexed with or covalently bound to at least one of the two individual polypeptide chains of the dimeric antigen-binding molecule. In one aspect, the further functional unit may be covalently bound to only one of the individual polypeptide chains and in another aspect the further functional unit may be covalently bound to both polypeptide chains of the dimeric antigen-binding molecule thereby linking the two polypeptide chains. In a further aspect, each of the two polypeptide chains is covalently bound individually to a further functional unit. When the further functional unit is covalently bound to at least one of the two polypeptide chains, the further functional unit may be fused to at least one of the two polypeptide chains by a peptide bond or a peptide linker. Alternatively, the further functional unit may be linked by a chemical conjugation such as a disulfide bridge, e.g. between a cysteine residue of at least one polypeptide chain and a cysteine residue of the further functional unit, ester linkage or by chemical crosslinking In a certain aspect of the invention the further functional unit may be linked to the antigen binding molecule by a cleavable linker such as, for example, a disulfide bond.

The further functional unit may be linked to the N-terminus or C-terminus of the first and/or second polypeptide chains. If one further functional unit is linked to both, the first and second, polypeptide chains, the further functional unit may be linked N-terminal to one polypeptide chain and C-terminal to the other polypeptide chain.

Homobifunctional and heterobifunctional reagents for chemical crosslinking of a polypeptide chain with a further functional unit such as a further polypeptide or an agent are well known in the art. Examples include but are not limited to 5,5′-dithiobis(2-nitrobenzoic acid) (DTNB), o-phenylenedimaleimide (o-PDM), succinimidyl 3-(2-pyridyldithio)propionate (SPDP), N-succinimidyl S-acetylthio acetate (SATA), succinimidyl 4-(N-maleimidomethyl)cyclohexane-1-carboxylate (SMCC) or 4-(4-N-maleimidophenyl)butyric acid hydrazide (MPBH). Methods for crosslinking of polypeptide chains comprising immunoglobulin chains with a further polypeptide or a chemical agent are described for example in Graziano et al., Methods in Molecular Biology, 2004, vol. 283, 71-85 and Hermanson, G. T. “Bioconjugate Techniques” Academic Press, London 1996.

In one aspect the further functional unit may be at least one further variable immunoglobulin domain. The further variable immunoglobulin domain may be specific for the first antigen A or the second antigen B for which the binding sites of the dimeric antigen-binding molecule are specific or, alternatively, specific for a third antigen C which is different from antigen A and antigen B. In a certain aspect a further light chain variable domain V_{L} and a further heavy chain variable V_{H} may be fused to each of the two polypeptide chains such that one further domain, in particular V_{H}, is fused to the N-terminus and the other further domain, in particular V_{L}, is fused to the C-terminus resulting in a polypeptide having six variable domains which will associate with another identical polypeptide to a dimeric antigen-binding molecule having six antigen-binding sites. In another aspect one further variable immunoglobulin domain may be fused to one of the polypeptide chains of the antigen-binding molecule which then non-covalently associates with a complementary variable immunoglobulin domain with the same specificity of a further third polypeptide thereby forming a further antigen-binding site between the dimeric antigen-binding molecule and the further third polypeptide. In another aspect a further antigen-binding unit including a scFv or a diabody may be linked as a further functional unit to the dimeric antigen-binding molecule.

In a certain aspect the further functional unit may be at least one further dimeric antigen-binding molecule as described herein. Accordingly, two or more dimeric antigen-binding molecules according to the invention may be linked with one another to increase the valency and avidity of the antigen binding molecules.

In another aspect the further functional unit may be an effector domain including Fc domain, CH2 domain, CH3 domain, hinge domain or a fragment thereof. Such a unit may confer effector properties on the antigen-binding molecule in the case of binding to Fc receptors. Such functional units may further be used to increase the serum-half life of the antigen-binding molecule.

In another aspect the further functional unit may be an enzyme. In the case where the enzyme is capable of converting a pro-drug to an active drug, such an antigen-binding molecule may be used in antibody-dependent enzyme prodrug therapy (ADEPT). For this the antigen-binding molecule directs the enzyme to the tissue of interest and when the antigen-binding molecule binds to the tissue, the prodrug is activated at that site. Further, the use of bispecific antigen-molecules for targeting enzymes for cancer therapeutics is known in the art, for example, but not limited to bispecific antigen-molecules having specificities for CD30 and alkaline phosphatase which catalyze the conversion of mitomycin phosphate to mitomycin alcohol, or specifities for placental alkaline phosphatase and β-lactamase which activate cephalosporin-based anti-cancer prodrugs. Suitable are also bispecific antigen-binding molecules having specificity for fibrin and tissue plasminogen activator for fibrinolysis and the use of enzyme conjugated antigen-binding molecules in enzyme-based immunoassays.

In another aspect the functional unit may be a drug, toxin, radioisotope, lymphokine, chemokine or labeling molecule. Such an antigen-binding molecule delivers the functional unit to the desired site of action. For example a chemotherapeutic drug linked to an antigen-binding molecule being specific for a tumor antigen can be delivered to a tumor cell and toxins may be delivered to pathogens or tumor cells. An antigen-binding molecule linked with a toxin may be used to target NK cells or macrophages and are preferably specific for CD16. Examples of a toxin are but not limited to ribosyl transferase, serine protease, guanyl cyclase activator, calmodulin dependent adenyl cyclase, ribunuclease, DNA alkylating agent or mitosis inhibitor, e.g. doxorubicin. The labeling molecule may be, for example, a fluorescent, luminescent or radiolabel molecule, a metal chelate or an enzyme (e.g. horse-radish peroxidase, alkaline phosphatase, β-galactosidase, malate dehydrogenase, glucose oxidase, urease, catalase etc.) which, in turn, when later exposed to a substrate will react to the substrate in such a manner as to produce a chemical moiety which can be detected and can be used for in vivo imaging or immunoassays, when it is linked to the antigen-binding molecule according to the invention. When used for an immunoassay, the dimeric antigen-binding molecule can also be immobilized on an insoluble carrier, e.g. glass, polystyrene, polypropylene, polyethylene, dextran, nylon, natural and modified celluloses, polyacrylamides, agarose and magnetic beads.

For increasing serum-half life of the antigen-binding molecules according to the invention in the body, the antigen-binding molecule, if desired, may be fused to albumin or pegylated, sialylated or glycosylated (see, for example, Stork et al., 2008, J. Biol. Chem., 283:7804-7812). Alternatively to a fusion of additional albumin to the antigen-binding molecule according to the present invention in some embodiments, the antigen-binding molecule itself may be specific for albumin and comprise light chain and heavy chain variable domains specific for albumin and , wherein albumin may represent antigen A or antigen B according to the invention. In a preferred embodiment, albumin may represent antigen A as illustrated by the construct of Example 2. Thus, in some embodiments the antigen-binding molecule according the present invention is specific for albumin and another antigen. Such antigen-binding molecules have an increased serum-half life. Such antigen-binding molecules comprise a polypeptide chain wherein the domains are arranged in the order V_{L}A-V_{H}B-V_{L}B-V_{H}A, wherein antigen A or antigen B is albumin.

The dimeric antigen-binding molecule according to any one of the embodiments described here previously may be produced by expressing polynucleotides encoding the individual polypeptide chains which associate with each other to form the dimeric antigen-binding molecule. Therefore, a further embodiment of the invention are polynucleotides, e.g. DNA or RNA, encoding the polypeptide chains of the dimeric antigen-binding molecule as described herein above.

The polynucleotides may be constructed by methods known to the skilled person, e.g. by combining the genes encoding the first domain V_{L}A, the second domain V_{H}B, the third domain V_{L}B and the fourth domain V_{H}A either separated by peptide linkers or directly linked by a peptide bond, into a single genetic construct operably linked to a suitable promoter, and optionally a suitable transcription terminator, and expressing it in bacteria or other appropriate expression system. Depending on the vector system and host utilized, any number of suitable transcription and translation elements, including constitutive and inducible promoters, may be used. The promoter is selected such that it drives the expression of the polynucleotide in the respective host cell.

The polynucleotides may be codon optimized with the codon bias being altered to suit the particular expression in the chosen host.

The polynucleotide may be inserted into vectors, preferably expression vectors, which represent a further embodiment of the invention. These recombinant vectors can be constructed according to methods well known to the person skilled in the art; see, e.g., Sambrook, Molecular Cloning A Laboratory Manual, Cold Spring Harbor Laboratory (1989) N.Y.

A variety of expression vector/host systems may be utilized to contain and express the polynucleotides encoding the polypeptide chains of the present invention. These include, but are not limited to, microorganisms such as bacteria transformed with recombinant bacteriophage, plasmid, or cosmid DNA expression vectors, yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, for which, e.g., viral-based expression systems may be utilised.

A particular preferred expression vector for expression in E. coli is pSKK (LeGall et al., J Immunol Methods. (2004) 285(1):111-27) or pcDNA5 (Invitrogen) for the expression in mammal cells.

Thus, the dimeric antigen-binding molecule as described herein may be produced by introducing a polynucleotide or vector encoding the polypeptide chain as described above into a host cell and culturing said host cell under conditions whereby the polypeptide chain is expressed. The dimeric antigen-binding molecule obtained from the expressed polypeptide chains may be isolated and, optionally, further purified. Conditions for the growth and maintenance of host cells, the expression, isolation and purification of dimeric antigen-binding molecules according to the invention from these host cells are fully described in the art.

In a further embodiment of the invention compositions comprising a dimeric antigen-binding molecule or a polynucleotide as described herein above and at least one further component are provided. For use in preventing or treating a disease or disorder the composition containing the dimeric antigen-binding molecule or the polynucleic acid molecule encoding the polypeptide chains forming the antigen-binding molecule is preferably combined with a suitable pharmaceutically acceptable carrier. The term “pharmaceutically acceptable carrier” is meant to encompass any carrier, which does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. Administration of the suitable compositions may be effected by different ways, e.g. by intravenous, intraperetoneal, subcutaneous, intramuscular, topical or intradermal administration. The route of administration, of course, depends on the kind of therapy and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind of therapy, general health and other drugs being administered concurrently.

The invention further provides a method wherein the dimeric antigen-binding molecule as described herein above is administered in an effective dose to a subject, e.g., patient, for the treatment of autoimmune disease, inflammatory disease, infectious disease, allergy or cancer (e.g. non-Hodgkin's lymphoma; chronic lymphocytic leukemia; Hodgkin's lymphoma; solid tumors e.g. those occurring in breast cancer, ovarian cancer, colon cancer, cancer of the kidney, or cancer of the bile duct; minimal residual disease; metastatic tumors e.g. those metastasizing in the lungs, bones, liver or brain). The antigen-binding molecule can be used in prophylactic or therapeutic settings, alone or in combination with current therapies.

The cancers that can be treated using the antigen-binding molecule of the present invention include but are not limited to primary and metastatic adrenal cortical cancer, anal cancer, aplastic anemia, bile duct cancer, bladder cancer, bone cancer, bone metastasis, CNS tumors, peripheral CNS cancer, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

An “effective dose” refers to amounts of the active ingredient that are sufficient to affect the course and the severity of the disease, leading to the reduction or remission of such pathology. An “effective dose” useful for treating and/or preventing these diseases or disorders may be determined using methods known to a skilled person (see for example, Fingl et al., The Pharmacological Basis of Therapeutics, Goddman and Gilman, eds. Macmillan Publishing Co., New York, pp. 1-46 (1975)).

In another aspect of the invention the dimeric antigen-binding molecule as described herein above is used in the manufacture of a medicament for the treatment of autoimmune disease, inflammatory disease, infectious disease, allergy or cancer (e.g. non-Hodgkin's lymphoma; chronic lymphocytic leukaemia; Hodgkin's lymphoma; solid tumours e.g. those occurring in breast cancer, ovarian cancer, colon cancer, cancer of the kidney, or cancer of the bile duct; minimal residual disease; metastatic tumours e.g. those metastasizing the lungs, bones, liver or brain). Where specified, multispecific binding molecules have been described above as having a particular utility in the treatment of a specified disease, these binding molecules may also be used in the manufacture of a medicament for that specified disease.

The methods for preparing pharmaceutical compositions, i.e. medicaments, and the clinical application of antigen binding molecules in the prevention and/or treatment of diseases such as, for example, cancer are known to the skilled artisan.

In a particular aspect of the invention the dimeric antigen binding molecule is bispecific and used for cancer therapy, because such antibodies can be used to retarget cytotoxic effector cells against tumor cells. This therapeutic concept is well known in the art. For example, clinical studies showed tumor regression in patients treated with an anti-CD3×antitumor bispecific antibody (e.g. Canevari, S. et al., J. Natl. Cancer Inst., 87:1463-1469,1996) or patients treated with an anti-CD16×antitumor bispecific antibody (e.g. Hartmann et al.; Clin Cancer Res. 2001;7(7):1873-81). Proof-of-concept has also been shown for various recombinant bispecific antibody molecules comprising only variable domains (Fv) such as, for example, dimeric and tetravalent CD3×CD19 antigen binding molecules having a domain order V_{H}A-V_{L}B-V_{H}B-V_{L}A (Cochlovius et al.; Cancer Research, 2000, 60:4336-4341)or recently in clinical studies with monomeric single-chain Fv antibody molecules of the BiTE®-format (two single-chain antibodies of different specificities linked together; Micromet A G, Germany; Bargou R. et al., Science, 2008, 321(5891):974-977; Baeuerle P A and Reinhardt C., Cancer Res. 2009, 69(12):4941-4944). The dimeric antigen binding molecules described herein can be used as medicaments and applied in methods of treatment in a similar way as the bispecific antibodies of the art, as they are capable of redirecting therapeutic, e.g. cytotoxic, mechanisms using the same combined antibody specificities. Further, immunosuppressive antibodies monospecific for CD3 such as Muromonab-CD3 are known for the treatment of transplant rejection, acute rejection of renal transplants (allografts), hepatic and cardiac transplants. Thus, antigen binding molecules bispecific for albumin and CD3 may be used in the same methods of treatments as the known monospecific anti-CD3 antibodies.

The antigen-binding molecule and the compositions thereof can be in the form of an oral, intravenous, intraperitoneal, or other pharmaceutically acceptable dosage form. In some embodiments, the composition is administered orally and the dosage form is a tablet, capsule, caplet or other orally available form. In some embodiments, the composition is parenteral, e.g. intravenous, intraperitoneal, intramuscular, or subcutaneous, and is administered by means of a solution containing the antigen-binding molecule.

A skilled person will readily be able without undue burden to construct and obtain the antigen-binding molecules described herein by utilizing established techniques and standard methods known in the art, see for example Sambrook, Molecular Cloning A Laboratory Manual, Cold Spring Harbor Laboratory (1989) N.Y.; The Protein Protocols Handbook, edited by John M. Walker, Humana Press Inc. (2002); or Antibody engineering: methods and protocols/edited by Benny K. C. Lo; Benny K. C. II Series: Methods in molecular biology (Totowa, N.J.)). In addition, a skilled person will be able to make the antigen-binding molecules described herein by utilizing standard methods known in the art and modifying the methods described in U.S. Pat. No. 7,129,330, Kipriyanov et al. J. Mol. Biol. (1999) 293, 41- 56 or Le Gall et al., 2004, Protein Engineering 17:357-366 such that dimeric antigen-binding molecules as described above comprising two polypeptide chains having the domain order V_{L}A-V_{H}B-V_{L}B-V_{H}A from the N-terminus to the C-terminus of each polypeptide chains are obtained.

The examples below further illustrate the invention without limiting the scope of the invention.

### EXAMPLE 1

To construct functional dimeric tandem diabodies (TandAb®) using a domain arrangement other than V_{H}A-V_{L}B-V_{H}B-V_{L}A, several such dimeric tandem diabodies were constructed with the domain arrangement V_{L}A-V_{H}B-V_{L}B-V_{H}A according to the invention using the two domains of a humanized anti-CD19 single chain antibody and a humanized anti-CD3 single chain antibody, respectively. The findings were confirmed by using two variants of each antigen-binding molecule, representing the products of different stages of an affinity maturation procedure that was carried out for both the humanized anti-CD19 and humanized anti-CD3 antibodies.

The murine monoclonal antibodies HD37 and UCHT directed against CD19 and CD3, respectively, were the starting material for obtaining humanized antibodies with relatively high affinities. In each case the V_{H} domain was first combined with a library of human V_{L} in an scFv phagemid vector to select a suitable human V_{L} chain by phage display. In a second step the selected human V_{L} chain was combined with a library of V_{H} domains in which the CDR3 region remained constant. This procedure resulted in a humanized anti CD19 and anti CD3, respectively, that only contained a short murine sequence in the V_{H}CDR3 region. These clones were subsequently affinity matured introducing point mutations at residues thought to be involved in antigen binding. The best binding mutants were then selected by phage display. The clones chosen for constructing the TandAb were M13 and M39 binding to CD19 and C4 and LcHC21 binding to CD3.

The following antibodies according to the invention were generated:

Antibody A1: CD19^{M39}×CD3^{C4} (option 0) V_{H}^{CD3C4}-V_{L}^{CD19M39}-V_{H}^{CD19M39}-V_{L}^{CD3C4}

Antibody B: CD19^{M39}×CD3^{C4} (option 2) V_{L}^{CD3C4}-V_{H}^{CD19M39}-V_{L}^{CD19M39}-V_{H}^{CD3C4}

Antibody A2: CD19^{M13}×CD3^{LCHC21} (option 0) V_{H}^{CD3LCHC21}-V_{L}^{CD19M13}-V_{H}^{CD19M13}- V_{L}^{CD3LCHC21}

Antibody C: CD19^{M13}×CD3^{LCHC21} (option 2) V_{L}^{CD3LCHC21}-V_{H}^{CD19M13}-V_{L}^{CD19M13}- V_{H}^{CD3LCHC21}

The plasmids encoding the hybrid monomers V_{L}^{CD3C4}-V_{H}^{CD19M39}-V_{L}^{CD19M39}-V_{H}^{CD3C4} of antibody B and V_{L}^{CD3LCHC21}-V_{H}^{CD19M13}-V_{L}^{CD19M13}-V_{H}^{CD3LCHC21} of antibody C were generated by a DNA engineering and processing provider. The sequence backbone of the V_{L}^{CD3C4}-V_{H}^{CD19M39}-V_{L}^{CD19M39}-V_{H}^{CD3C4} monomer comprises the DNA sequences of two scFv antibodies, namely scFvCD19^{M39} and scFvCD^{C4}, respectively. the V_{L}^{CD3LCHC21}-V_{H}^{CD19M13}-V_{L}^{CD19M13}- V_{H}^{CD3LCHC21} monomer sequence combines the variable domains of the single chain Fv CD19^{M13} and single chain Fv CD3^{LCHC21}. All four scFv were obtained by phage display selection of single chain antibodies against the antigens CD19 and CD3. In both cases the sequence information was used to construct the above hybrid monomers. A 9 amino acid (G₂S)₃ linker was used to link the domains with one another. The synthesized gene coding for V_{L}^{CD3C4}-V_{H}^{CD19M39}-V_{L}^{CD19M39}-V_{H}^{CD3C4} was cloned into the mammalian expression vector pCDNA5FRT (Invitrogen). The gene of V_{L}^{CD3LCHC21}-V_{VH}^{CD19M13}-V_{L}^{CD19M13}-V_{H}^{CD3LCHC21} was also cloned into an expression vector and amplified by PCR using a forward primer introducing an NcoI cleaving site and a reverse primer introducing a NotI cleaving site. After analysis and isolation by agarose gel, the PCR product was subsequently double digested by NcoI and NotI and cloned into the NcoI and NotI linearised pSKK3 vector. The correct cloning was confirmed by DNA sequencing.

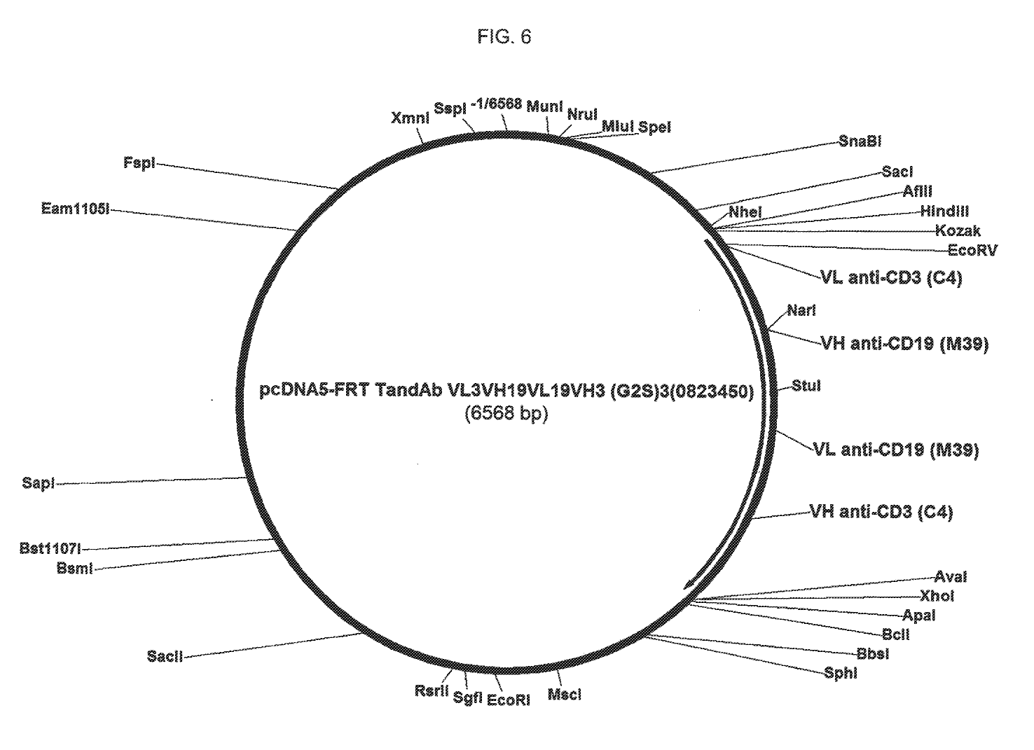

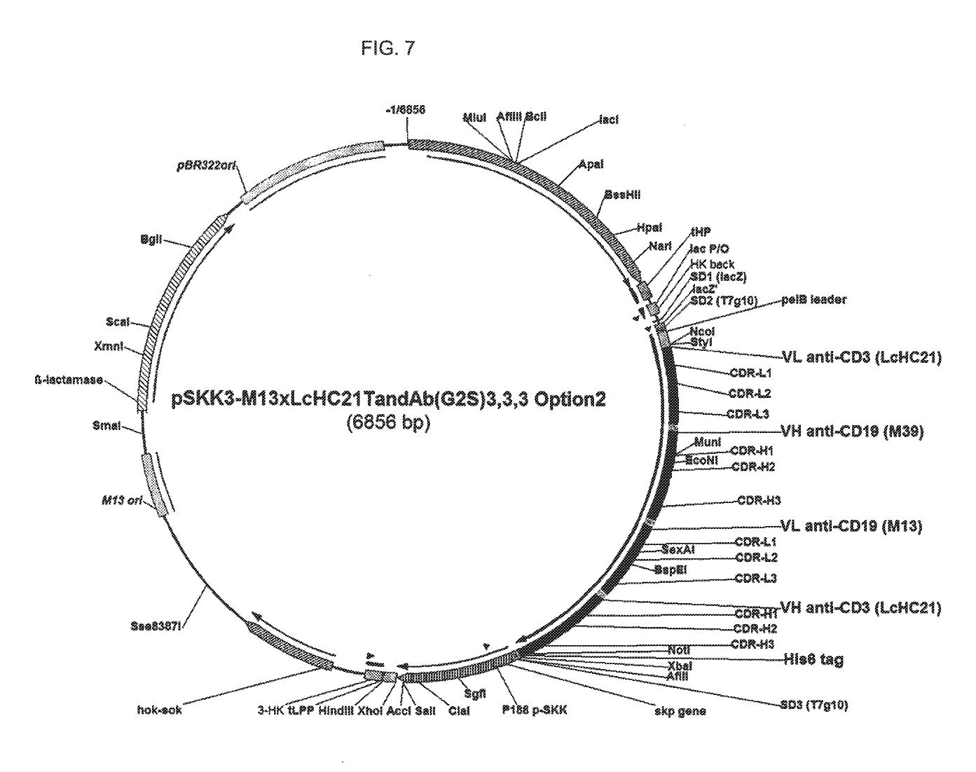

The vector map of pCDNA5FRT encoding antibody B is shown in FIG. 6. The vector map of pSKK3 encoding antibody C is shown in FIG. 7.

For high level production the vector containing the gene V_{L}^{CD3C4}-V_{H}^{CD19M39}-V_{L}^{CD19M39}-V_{H}^{CD3C4} was transiently transfected (using CaPO₄) into adherent HEK293 cells. Protein fermentation was performed under growth conditions well known in the art.

The recombinant protein was expressed as a His-Tag fusion protein with a signal peptide. The protein was isolated from cell culture supernatant by immobilized metal affinity chromatography (IMAC) as described (Kipriyanov et al., 1999, J.Mol.Biol., 293, 41-56). The purified material was subsequently analysed by SDS-PAGE. Coomassie staining of an SDS PAGE gel and size-exclusion chromatography on a calibrated Superdex 200 HR10/30 column (Amersham Pharmacia, Freiburg, Germany) in sodium-phosphate buffer (30 mM NaPO₄, 0.75 M arginine/HCl, pH 6.0) revealed a pure and correctly assembled recombinant protein (Antibody B).

For high level expression, the gene coding for the humanized V_{L}^{CD3LCHC21}-V_{H}^{CD19M13}-V_{L}^{CD19M13}-V_{H}^{CD3LCHC21} monomer followed by a 6× His-Tag was cloned into the pSKK3 plasmid containing the hok/sok gene cell suicide system and a skp gene encoding the Skp/OmpH periplasmic factor (LeGall et al., 2004, J. Immunol. Methods, 285, 111-127). The plasmid was was transfected into an E. coli K12 strain (ATCC 31608™).

The transformed bacteria were grown in shake flasks and induced essentially as described previously (Cochlovius et al., 2000, J. Immunol., 165, 888-895). The recombinant proteins were isolated from both the soluble periplasmic fraction and the bacterial medium supernatant by immobilized metal affinity chromatography (IMAC) as already described (Kipriyanov et al., 1999, J.Mol.Biol., 293, 41-56).

The purified material was subsequently analysed by SDS-PAGE stained by Coomassie blue and size-exclusion chromatography on a calibrated Superdex 200 HR10/30 column (Amersham Pharmacia, Freiburg, Germany) in sodium-phosphate buffer (30 mM NaPO₄, 0.75 M arginine/HCl, pH 6.0). The product appeared to be pure and correctly assembled.

The comparative antibodies A1 and A2 were generated in the same way as antibodies B and C, respectively, wherein the domain order of antibodies A1 and A2, respectively, were reversed in comparison to that of antibodies B and C, respectively.

Cytotoxicity assays were performed essentially as described by T. Dreier et al. (2002, Int J Cancer 100, 690-697). The PMBCs that were used as effector cells were isolated from the peripheral blood of healthy volunteers by density gradient centrifugation. In some cases, the PBMC were cultured overnight in the presence of 25 U/mL human IL-2 before they were used as effector cells in the cytotoxicity assay. Purity and antigen expression of the isolated PBMC was checked by flow cytometry in each case (data not shown).

CD19 ⁺ JOK-1 or Raji target cells were cultured in RPMI 1640 medium supplemented with 10% FCS, 2 mM L-glutamine and 100 IU/mL penicillin G sodium and 100 μg/mL streptomycin sulfate (herein referred to as RPMI medium; all components from Invitrogen). For the cytotoxicity assay cells were labeled with 10 μM calcein AM (Molecular Probes/Invitrogen) for 30 min in RPMI medium without FCS at 37° C. After gently washing the labeled cells were resuspended in RPMI medium to a density of 1×10⁵/mL. 1×10⁴ target cells were then seeded together with 5×10⁵ PBMC with the indicated antibodies in individual wells of a round-bottom 96-well micro plate in a total volume of 200 μL/well. After centrifugation for 2 min at 200 g the assay was incubated for 4 hours at 37° C. in a humidified atmosphere with 5% CO₂. 15 min prior to the end of incubation 20 μL of 10% Triton X-100 in RPMI medium were added to the wells with target cells only. 20 μL RPMI medium was added to all other wells. 100 μL cell culture supernatant were harvested from each well after an additional centrifugation for 5 min at 500 g, and the fluorescence of the released calcein was measured at 520 nm using a fluorescence plate reader (Victor 3, Perkin Elmer). On the basis of the measured counts, the specific cell lysis was calculated according to the following formula: [fluorescence (sample)−fluorescence (spontaneous)]/[fluorescence (maximum)−fluorescence (spontaneous)]×100%. Fluorescence (spontaneous) represents the fluorescent counts from target cells in the absence of effector cells and antibodies and fluorescence (maximum) represents the total cell lysis induced by the addition of Triton X-100. Sigmoidal dose response curves and EC₅₀ values were calculated using the Prism software (GraphPad Software).

### Results

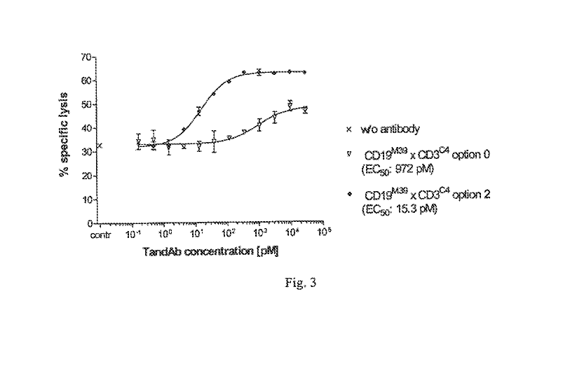

The results of the cytotoxicity assays for tandem diabodies having the following domain order starting at the N-terminus of V_{H}A-V_{L}B-V_{H}B-V_{L}A (antibody A) and V_{L}A-V_{H}B-V_{L}B-V_{H}A (antibody B), respectively, using the anti CD19 variant M39 and the anti CD3 variant C4 are shown in FIG. 3.

Surprisingly, there was a very large difference in the cytotoxic activity of the two tandem diabodies. The tandem diabody having the domain arrangement according to the invention designated as “antibody B” was more than 60× more active than the tandem diabody designated “antibody B” as determined by a comparison of their EC₅₀ values under the given conditions.

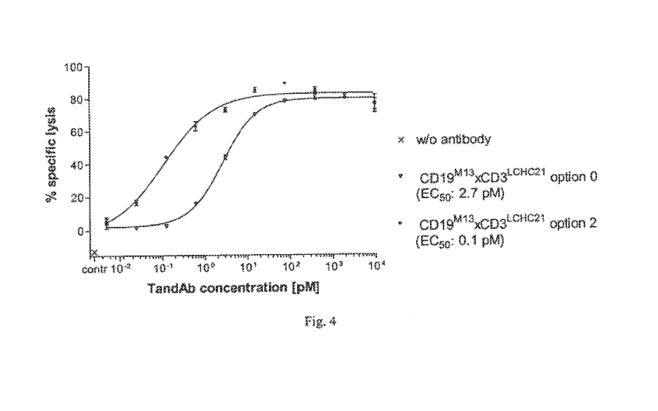

The superiority of the domain arrangement represented by the present invention (antibody C) for better cytotoxicity was confirmed by using two additional variants of the anti CD19 and anti CD3 antibodies (see FIG. 4).

The EC₅₀ value of the tandem diabody with the domain order according to the invention represented by option 2 is extremely low (0.1 pM). It is 27× more active than the TandAb represented by option 0 after comparing the EC₅₀ values under the given conditions.

### EXAMPLE 2

Human Serum Albumin (HSA)×CD3 TandAb

T cell receptor modulation by HSA×CD3 TandAb antibodies in vitro

To determine whether the HSA×CD3 TandAb antibodies with different domain orders differ in efficacy in inducing T cell receptor (TCR)/CD3 modulation on T cells in vitro CD3⁺ Jurkat cells were cultured in the presence of increasing concentrations of the bispecific HSA×CD3 TandAb antibodies and subsequently analyzed for remaining TCR. The modulation assay was performed in the presence or absence of HSA to measure the influence of HSA on the activity of the TandAbs.

In brief, 1×10⁶ Jurkat cells were seeded in individual wells of a round-bottom 96-well micro plate in RPMI 1640 medium supplemented with 2 mM L-glutamine and 100 IU/mL penicillin G sodium and 100 μg/mL streptomycin sulfate (all components from Invitrogen). In a separate micro plate Jurkat cells were seeded in RPMI medium as described before but with the addition of 50 mg/mL HSA (Sigma). After the addition of the indicated antibodies, cells were incubated in a total volume of 200 μL/well at 37° C. in a humidified incubator in the presence of 5% CO₂. As a control, cells were cultured in the absence of antibodies. After washing with ice-cold phosphate buffered saline (PBS, Invitrogen, Karlsruhe, Germany) supplemented with 2% heat-inactivated FCS (Invitrogen, Karlsruhe, Germany) and 0.1% sodium azide (Roth, Karlsruhe, Germany) (referred to as a FACS buffer) the cells were stained with 10 μL PC5-conjugated anti-TCR α/β antibody (Beckman-Coulter) in a total volume of 100 μL in FACS buffer for 45 on ice in the dark. After washing twice with FACS buffer the fluorescence of 10⁴ cells was measured at 675 nm with an FC500 MPL flow cytometer (Beckman-Coulter). Mean fluorescence values were determined using the CXP software (Beckman-Coulter) and used for analysis by non-linear regression/4 parameter logistic fit using the GraphPad Prism version 3.03 for Windows, GraphPad Software, San Diego Calif. USA.

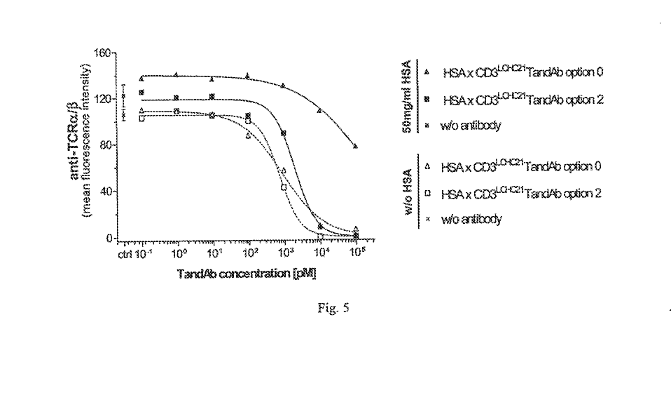

The results obtained from the TCR modulation experiment CAB-306 depicted in FIG. 5 and summarized in Tab. 1 demonstrate comparable TCR modulation efficacy of HSA×CD3 TandAb in domain order V_{H}^{HSA}-V_{L}^{CD3}-V_{H}^{CD3}-V_{L}^{HSA} (=option 0 (V_{H}A-V_{L}B-V_{H}B-V_{L}A)) and V_{L}^{HSA}-V_{H}^{CD3}-V_{L}^{CD3}-V_{H}^{HSA} (=option 2 (V_{L}A-V_{H}B-V_{L}B-V_{H}A)). However, in the presence of physiological concentrations of HSA the modulation efficacy in case of the option 0 (V_{H}A-V_{L}B-V_{H}B-V_{L}A) TandAb is considerably decreased, whereas the EC₅₀ value for the TandAb in the option 2 (V_{L}A-V_{H}B-V_{L}B-V_{H}A) orientation is only increased by factor 2.6.

These data clearly indicated the superior properties of the HSA×CD3 TandAb in domain orientation option 2 (V_{L}A-V_{H}B-V_{L}B-V_{H}A) when compared with the HSA×CD3 TandAb option 0 (V_{L}A-V_{H}B-V_{L}B-V_{H}A).

TABLE 1
Summary of the results from the TCR modulation experiment: The EC₅₀
values from the TCR modulation experiment with the two HSA × CD3 TandAb
antibodies in the presence or absence of HSA (FIG. 5; experiment CAB-306) were
determined by non-linear regression/4 parameter logistic fit.

| TandAb antibody | | | EC₅₀ w/o | EC₅₀ with | fold increase |
|---|---|---|---|---|---|
| batch | TandAb | domain order | HSA | HSA | in EC₅₀ |
| MST13.1 | HSA × CD3 | option 0 | 861 pM | ~140000 pM | >100 |
| | | V_{H}A-V_{L}B-V_{H}B-V_{L}A | | | |
| MST13.3 | HSA × CD3 | option 2 | 726 pM | 1913 pM | 2.6 |
| | | V_{L}A-V_{H}B-V_{L}B-V_{H}A | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A dimeric antigen-binding molecule consisting of a first and a second polypeptide chain, each of the first and the second polypeptide chains comprising
a first domain V_{L}A being a light chain variable domain specific for a first antigen A;
a second domain V_{H}B being a heavy chain variable domain specific for a second antigen B;
a third domain V_{L}B being a light chain variable domain specific for the second antigen B; and
a fourth domain V_{H}A being a heavy chain variable domain specific for the first antigen A, wherein
the first domain V_{L}A and the fourth domain V_{H}A are specific for CD3;
the second domain V_{H}B and the third domain V_{L}B are specific for a tumor cell;
said domains are arranged in each of said first and second polypeptide chains in the order V_{L}A-V_{H}B-V_{L}B-V_{H}A from the N-terminus to the C-terminus of said polypeptide chains, and
the first domain V_{L}A of the first polypeptide chain is in association with the fourth domain V_{H}A of the second polypeptide chain to form an antigen binding site for the CD3;
the second domain V_{H}B of the first polypeptide chain is in association with the third domain V_{L}B of the second polypeptide chain to form an antigen binding site for the tumor cell;
the third domain V_{L}B of the first polypeptide chain is in association with the second domain V_{H}B of the second polypeptide chain to form an antigen binding site for the tumor cell; and
the fourth domain V_{H}A of the first polypeptide chain is in association with the first domain V_{L}A of the second polypeptide chain to form an antigen binding site for the CD3.

2. The antigen-binding molecule according to claim 1, wherein the first and the second polypeptide chains are non-covalently associated.

3. The antigen-binding molecule according to claim 1, wherein the antigen-binding molecule is tetravalent.

4. The antigen-binding molecule according to claim 1, wherein the domains are human domains or humanized domains.

5. The antigen-binding molecule according to claim 1, wherein said antigen-binding molecule comprises at least one further functional unit.

6. A polypeptide chain consisting of
a first domain V_{L}A being a light chain variable domain specific for a first antigen A;
a second domain V_{H}B being a heavy chain variable domain specific for a second antigen B;
a third domain V_{L}B being a light chain variable domain specific for the second antigen B; and
a fourth domain V_{H}A being a heavy chain variable domain specific for the first antigen A,
wherein the domains are arranged in the polypeptide chain in the order V_{L}A-V_{H}B-V_{L}B-V_{H}A from the N-terminus to the C-terminus of the polypeptide chains.

7. The polypeptide chain according to claim 6, wherein the domains are specific for CD3 and CD19.

8. A pharmaceutical composition comprising the antigen-binding molecule according to claim 1 and a pharmaceutically acceptable carrier.

9. A method for immunotherapy comprising the step of administering the composition of claim 8.
